(19) United States
(12) Patent Application Publication
(10) Pub. No.: US 2021/0038491 A1
(43) Pub. Date: Feb. 11, 2021

(54) Composition for Inactivating Smell Component

(71) Applicant: MORINAGA MILK INDUSTRY CO., LTD., Tokyo (JP)

(72) Inventors: Manabu Nakano, Kanagawa (JP); Hiroyuki Wakabayashi, Kanagawa (JP)

(73) Assignee: MORINAGA MILK INDUSTRY CO., LTD., Tokyo (JP)

(21) Appl. No.: 17/040,676

(22) PCT Filed: Mar. 15, 2019
(86) PCT No.: PCT/JP2019/010820

§ 371 Date: Sep. 23, 2020

(30) Foreign Application Priority Data
Mar. 23, 2018 (JP) 2018-056397

(51) Int. Cl. A61K 8/66; A61K 8/60; A61K 8/46; A61Q 11/00; A61Q 15/00

(52) U.S. Cl. CPC A61K 8/66; A61K 8/60; A61Q 15/00; A61Q 11/00; A61K 8/46

(57) ABSTRACT

Provided is a composition and a method capable of inactivating a generated smell component. A composition for inactivating a smell component, which contains lactoperoxidase, glucose oxidase, and glucose, and is used in the presence of thiocyanic acid or a salt thereof; it is desirable that the smell component is food-derived and/or smoking-derived; and it is desirable that the food-derived smell component is a smell component derived from a food including a plant of the genus Allium.

### TECHNICAL FIELD

The present invention relates to a composition for inactivating a smell component and a method of inactivating a smell component.

### BACKGROUND ART

Plants of the genus Allium such as garlic, green onions, leeks, and onions are often used for foods because their unique scent or taste stimulates the appetite, and they are said to be good for the body. Also, it is also recommended that fish be actively ingested for the purpose of maintaining health or promoting health. Meanwhile, cigarettes are smoked as favorite goods.

However, in many cases, a component such as a sulfur component or a nitrogen component (for example, allicin, choline, etc.) present in plants of the genus Allium, fish, or cigarettes becomes a causative substance (a starting substance, an intermediate substance, etc.) that changes into a smell component. The causative substance changes into an unpleasant smell component in the body by microorganisms or heat. Then, this unpleasant smell component is released to the outside of the body.

As this unpleasant smell component, hydrogen sulfide; volatile sulfur compounds of mercaptans such as methyl mercaptan or allyl mercaptan; ammonia; and volatile nitrogen compounds of amines such as trimethylamine, or nicotine (pyridine-based) are known. Then, this unpleasant smell component is generated from the breath immediately after eating, and may be generated from breathing, skin, or the like until the next day if the unpleasant smell component is present in the blood flow in the body.

In recent years, as the awareness of etiquette increases, there is a concern about smells emitted from animals (for example, humans, pets, etc.), such as a bad breath or a body odor, or an excretion smell, such as a urine smell or a fecal smell. Thus, there is a demand to deodorize or prevent an unpleasant smell such as bad breath or body odor.

As a conventional technology, there is a technology of targeting microorganisms or enzymes so as to suppress the production of a smell component through a sterilizing action or an enzyme activity inhibiting action. Such a conventional technology is a technology in which a smell component that is already generated is not inactivated by decomposition or the like (for example, sterilization, enzyme activity inhibition, etc.), but a stage before a causative substance changes into the smell component (that is, a production stage) is suppressed. Also, as conventional technologies, there are a technology of modulating smell by fragrance, and a technology of adsorbing a smell component.

For example, the technology of suppressing generation of a smell component may include: an oral germicide for sterilization in the oral cavity by a lactoperoxidase system (Patent Literature 1); an oral composition containing an olive leaf extract as an active ingredient, which suppresses methyl mercaptan generation by antibacterial activity of Porphyromonas gingivalis (Pg bacteria) of Periodontal disease pathogen (Patent Literature 2); a hydrogen sulfide enzyme inhibitor that has catechins as an active ingredient (Patent Literature 3); and a sulfur-containing amino acid lyase inhibitor using a lactoperoxidase system (Patent Literature 4).

### CITATION LIST

### Patent Literature

Patent Literature 1: WO2008/105113

Patent Literature 2: JP-A-2016-147869

Patent Literature 3: JP-A-2011-051946

Patent Literature 4: JP-A-2015-149904

### SUMMARY OF INVENTION

### Technical Problem

A smell component that is already produced is released to the outside of the body, as a bad breath or a body odor. However, there is a demand to avoid release from one's self or others this smell component released as bad breath or body odor as much as possible.

For example, immediately after garlic is eaten, allyl mercaptan is present in bad breath, and the allyl mercaptan is generated in digestive organs such as a stomach or in an oral cavity. Further, an unpleasant smell component or a causative substance thereof is digested and absorbed in the body, and is moved to each organ through a blood flow. The causative substance gradually changes into a smell component by metabolism or the like while moving through the blood flow.

As a result, the smell component is released as bad breath to the outside of the body from a stomach or a lung, or the smell component that changed in the oral cavity is released through the breath to the outside of the body. Also, the unpleasant smell component that is included in the blood flow or is changed on skin is released as a body odor, from the skin to the outside of the body. Also, an intestinal gas containing the unpleasant smell component, urination, defecation or the like may be released as an excretion smell (a urine smell, a fecal smell, etc.) to the outside of the body, which may cause an unpleasant odor.

Here, the “bad breath” is a smell or odor of an oral cavity or exhalation during breathing.

Also, there are relatively stable substances (for example, mercaptan, trimethylamine, etc.) in the generated smell component. It is thought that for these relatively stable substances, it is difficult to chemically adsorb the smell component or to decompose the smell component. As described above, it is generally said that it is technically difficult to inactivate and deodorize the generated smell component or to reduce the smell.

Therefore, a main object of the present technology is to provide a composition and a method capable of inactivating a generated smell component.

### Solution to Problem

The inventors have conducted intensive studies by using a model that directly inactivates a generated smell component itself as illustrated in Examples below.

Meanwhile, it is generally known that when methyl mercaptan is treated with a hydrogen peroxide solution which is often used as a germicide or a bleach, 2CH₃SH+HOOH→CH₃S—SCH₃ (methyl disulfide)+2H₂O. Since the methyl disulfide is also a smell component, it is not possible to inactivate a smell component even by using the hydrogen peroxide solution.

However, as a result of intensive studies, the inventors totally unexpectedly have found that an LPO reaction system using lactoperoxidase, glucose oxidase, and glucose may reduce a smell (for example, bad breath, body odor, excrement smell, etc.) by directly inactivating the smell component itself in the presence of thiocyanic acid or a salt thereof, and have completed the present invention.

Further, the present technology is a technology that targets the smell component itself, and is a technology having a completely different system from a conventional technology.

That is, the present invention is as follows.

The present technology provides a composition for inactivating a smell component comprising lactoperoxidase, glucose oxidase, and glucose, which is used in the presence of thiocyanic acid or a salt thereof.

The present technology provides a smell-component inactivating composition kit comprising lactoperoxidase, glucose oxidase, and glucose, which is used in the presence of thiocyanic acid or a salt thereof.

The present technology provides a method of inactivating a smell component by using lactoperoxidase, glucose oxidase, and glucose, in the presence of thiocyanic acid or a salt thereof.

The above smell component may be a volatile sulfur compound and/or a volatile nitrogen compound.

The above smell component may be food-derived and/or smoking-derived.

The above food-derived smell component may be a smell component derived from a food including a plant of the genus Allium.

The above composition kit may include at least the following (a), (b), (c), or (d). (a) a composition containing lactoperoxidase, a composition containing glucose oxidase, and a composition containing glucose; (b) a composition containing lactoperoxidase and glucose oxidase, and a composition containing glucose; (c) a composition containing lactoperoxidase, and a composition containing glucose oxidase and glucose; (d) a composition containing lactoperoxidase and glucose, and a composition containing glucose oxidase.

### Advantageous Effects of Invention

According to the present technology, it is possible to provide a composition and a method capable of inactivating a generated smell component. The effect described herein is not necessarily limited, and may be any of effects described in the present technology.

### DESCRIPTION OF EMBODIMENTS

Next, a preferred embodiment of the present invention will be described. Meanwhile, the present invention is not limited to the following preferred embodiment, and may be freely changed within the scope of the present invention. Also, in this specification, the percentage is expressed by mass unless otherwise specified.

The present technology is a smell-component inactivating composition that includes lactoperoxidase, glucose oxidase, and glucose, which is used in the presence of thiocyanic acid or a salt thereof.

Also, the present technology is a composition kit that includes lactoperoxidase, glucose oxidase, and glucose, which inactivates a smell component in the presence of thiocyanic acid or a salt thereof.

Also, the present technology is a method of inactivating a smell component by using lactoperoxidase, glucose oxidase, and glucose in the presence of thiocyanic acid or a salt thereof.

An enzyme reaction system that uses lactoperoxidase, glucose oxidase, and glucose in the present technology is also referred to as an “LPO reaction system.”

In the present technology, an application target may be an animal, and also, an application target may be a substance in which a smell component is generated.

The animal as the application target may include humans, and animals other than humans (preferably, mammals, birds, reptiles, etc.). Among them, humans and pets are preferred, and humans are more preferred.

Also, the application target object may include materials including smell components, such as, for example, foods, clothes, cloth products, sanitary products, excrements, smell-attached products.

Also, the present technology may be used for a therapeutic purpose, or may be used for a non-therapeutic purpose. The “non-therapeutic purpose” is a concept that does not include a medical act, that is, an act of treating a human body by treatment. A bad breath etiquette or the like may be exemplified.

“Prevention” refers to prevention or delay of the onset of a disease or a symptom in the application target, or risk reduction of a disease or a symptom of the application target.

“Improvement” refers to improvement of a disease, a symptom or a condition; prevention or delay of deterioration; or reversal, prevention or delay of progression.

Components of the present technology, that is, lactoperoxidase, glucose oxidase, and glucose, and thiocyanic acid or a salt thereof, will be described in detail by the following (1) to (4).

(1) Component (A) Lactoperoxidase

It is generally known that Lactoperoxidase is oxidoreductase in milk and has a function of catalyzing generation of hypothiocyanic acid and water from hydrogen peroxide and thiocyanic acid.

The lactoperoxidase used in the present technology is not particularly limited, but it is desirable to use one derived from mammalian milk. Among the corresponding lactoperoxidases, lactoperoxidases derived from milk of cows, horses, sheep, goats, etc. are preferred, and one derived from milk of cows is more preferred.

Since milk has been used for foods and drinks of humans for many years, lactoperoxidase derived from milk is also highly safe for animals such as humans, and thus the milk-derived lactoperoxidase is preferred. Further, from the viewpoint of safety based on eating experience, and large-scale stable productivity, lactoperoxidase derived from cow milk is more preferred.

The above lactoperoxidase may be obtained from mammalian milk or the like, and may be obtained from milk of, for example, humans, cows, horses, sheep, goats, etc.

It is desirable that lactoperoxidase used in the present technology is industrially produced from unheated whey such as milk, or skim milk, according to a conventional method (for example, ion exchange chromatography, etc.) (for example, Reference Literature 1 (Japanese Laid-Open Patent Publication No. 5-41981), and Reference Literature 2 (WO2005/078078)).

Also, commercially available natural product-derived lactoperoxidase (manufactured by, for example, Biopole), recombinant lactoperoxidase, expressed purified recombinant lactoperoxidase (for example, Reference Literature 3 (Biochemical and Biophysical Research Communications, volume 271, 2000, p. 831-836)), or commercially available recombinant lactoperoxidase may be used.

Among these, cow milk-derived skim milk or whey is preferred as a raw material of lactoperoxidase to be used in the present technology, in view of stably obtaining a large amount.

(2) Component (B) Glucose Oxidase

Glucose oxidase is generally known as an enzyme that oxidizes β-D-glucose to produce D-glucono-δ-lactone and hydrogen peroxide.

The glucose oxidase used in the present technology is not particularly limited, but glucose oxidase derived from microorganisms is preferred in view of quality, stability, and productivity.

Examples of the corresponding microorganism-derived enzyme may include enzymes produced by microorganisms such as Aspergillus niger and Penicillium chrysogenum.

The corresponding microorganism-derived glucose oxidase may be obtained by using a conventionally known method for producing microorganism-derived enzymes. Also, commercially available glucose oxidase may be used, or commercially available microorganism-derived glucose oxidase (manufactured by, for example, Shin Nippon Chemical Industry Co., Ltd.) may be used.

(3) Component (C) Glucose

Glucose used in the present technology is not particularly limited, and, for example, commercially available glucose for food additives (manufactured by, for example, Nippon Food Chemical Co., Ltd.) may be used. Also, glucose-containing products, for example, isomerized sugar, starch syrup, starch hydrolysate, etc. may be used.

(4) Component (D) Thiocyanic Acid or Salt Thereof

The present technology is used in the presence of thiocyanic acid or a salt thereof. When the corresponding thiocyanic acid or the salt thereof is added as necessary, commercially available products (manufactured by, for example, Merck Millipore) may be used. The salt is not particularly limited, and may include alkali metal salts (for example, sodium salt, potassium salt, etc.), iron (III) salt, and the like.

When the LPO reaction system of the present technology is used in a region where thiocyanic acid is already present (for example, in the oral cavity), the thiocyanic acid or the salt thereof may not be added, and also, the thiocyanic acid or the salt thereof may not be present in the composition of the present technology.

When the LPO reaction system of the present technology is used in a region where thiocyanic acid or a salt thereof is not present, it is desirable to separately add the thiocyanic acid or the salt thereof to the region, and also desirable to add the thiocyanic acid or the salt thereof in the LPO reaction system or the composition of the present technology.

Also, when the amount of the thiocyanic acid or the salt thereof of the present technology is insufficient in the region where the LPO reaction system is reacted, it is desirable to separately add the insufficient amount to the LPO reaction system or the region, and it is also desirable to add the insufficient thiocyanic acid or the salt thereof to the composition of the present technology.

It is desirable that the composition of the present technology further contains a pH adjusting component, in view of carrying out a stable reaction. Also, the composition of the present technology may be properly blended with optional components within a range in which the effect of the present technology is not impaired.

The pH adjusting component of the composition of the present technology is not particularly limited, but preferably has a pH of 4.0 to 9.0, more preferably 6.0 to 8.0, further preferably 7.0 to 8.0 when dissolved in an aqueous solvent.

Examples of the corresponding pH adjusting component may include inorganic acids, organic acids and salts thereof, and these may be used alone or in combination of two or more types. It is desirable that the corresponding pH adjusting component is water-soluble, and also commercially available food additives may be used.

The above inorganic acid may include phosphoric acid, nitric acid, sulfuric acid, and the like.

Examples of the above organic acid may include citric acid, lactic acid, malic acid, succinic acid, tartaric acid, glutamic acid, etc.

The salt may include alkali metal salts (lithium, potassium, sodium, etc.), alkaline earth metal salts (calcium, magnesium, etc.), and the like.

One type or two or more types of the above pH adjusting components may be used in combination.

In the present technology, the “smell component” itself as a target may react with the above described LPO reaction system, and thus the corresponding smell component may be inactivated. The mechanism of inactivation of the smell component is being currently intensively investigated, but as illustrated in the Examples below, it may be thought that at least the LPO reaction system is directly or indirectly involved in inactivation of the smell component itself. By this inactivation, odor caused by the smell component may be reduced or eliminated.

The “smell component” that is a target of the present technology will be described below in detail.

The “smell component” as a target of the present technology is not particularly limited, but examples thereof may include smell components generated due to foods and/or smoking.

In the present technology, a food-derived smell component and/or a smoking-derived smell component is preferred. These contain an unpleasant smell component to be described below or a causative substance thereof.

When an animal ingests food or the animal smokes cigarettes, an unpleasant smell component caused by the food and/or smoking, or the corresponding causative substance is present in the body. Smoking also includes indirect smoking. Then, the unpleasant smell component is released to the outside of the body by breathing, skin breathing, excretion or the like.

A “bad breath-derived smell component” may include: a smell component generated in a stomach space (hereinafter, also referred to as a “stomach-derived smell component”); a smell component released from a lung when the smell component is present in a blood flow (hereinafter, also referred to as a “lung-derived smell component”); and a smell component generated in the oral cavity (hereinafter, also referred to as an “oral cavity-derived smell component”).

These stomach-derived, lung-derived, and oral cavity-derived smell components are included in the breath. Thus, a gas containing these generated smell components is released as breath through breathing. This breath is recognized as “bad breath.”

Also, a “body odor-derived smell component” may include: a smell component released by skin breathing when the smell component is present in a blood flow (hereinafter, also referred to as a “skin breathing-derived smell component”); and a smell component generated by a change of secretions (such as sweat) on skin (hereinafter, also referred to as a “skin secretion-derived smell component”).

A gas containing these skin-derived smell components is released from skin. This gas is recognized as a “body odor.”

Also, an “excretion-derived smell component” may include: a smell component derived from an intestinal gas; and a smell component derived from feces and urine. A gas containing these excretion-derived smell components is recognized as a “urine smell, fecal smell.”

According to the present technology, a smell component released to the outside of the body may be inactivated. The present technology is preferably effective for a smell component contained in bad breath or body odor.

Also, in the present technology, since an oral cavity is a narrow space and is a place moved by mastication or the like, the LPO reaction system of the present technology easily reacts with a smell component present in the oral cavity or a smell component passing through the oral cavity. Accordingly, it is easy for the LPO reaction system of the present technology to easily and immediately inactivate the corresponding smell component, and thus the bad breath may be immediately, efficiently, and simply reduced or eliminated.

The “smell component” as a target of the present technology is preferably a bad breath-derived smell component, a body odor-derived smell component, or an excretion-derived smell component.

The corresponding bad breath-derived smell component is preferably a stomach-derived smell component or a lung-derived smell component. The stomach-derived smell component is a smell component that is released to the outside of the body due to the presence of a food or smoking component in a stomach.

Also, the lung-derived smell component is a smell component that is released to the outside of the body because a causative substance or a smell component generated by food ingestion or the like is present in a blood flow.

Also, the above body odor-derived smell component is preferably a skin-derived smell component, and is a smell component that is released to the outside of the body due to the presence of a causative substance or the smell component in a blood flow or on skin.

Also, the above excretion-derived smell component is a smell component when a causative substance or a smell component generated by food ingestion or the like is released from a gas or excrements such as feces and urine.

The “smell component” as a target of the present technology is preferably caused by food having a smell component and a causative substance thereof, and is released to the outside of the body due to the corresponding food in many cases.

As for the food having the above causative substance, plants of the genus Allium may be exemplified. Examples of the corresponding plants of the genus Allium may include green onions, rakkyo, garlic, onions, leeks, and the like. One type or two or more types selected from these are often used as food raw materials. These contain a large amount of causative substance that becomes mercaptan.

Therefore, in the present technology, it is possible to inactivate a food-derived smell component which occurs when the corresponding food including a plant of the genus Allium is eaten. In the conventional technology, it was not possible to inactivate a smell component derived from the plant of the genus Allium, which is generated in the body (particularly, after digestion and absorption). For example, although there is bad breath or body odor generated the day after a plant of the genus Allium is eaten, it was not possible to deodorize such a smell using the conventional technology. Meanwhile, in the present technology, it is possible to inactivate a generated smell component emitted as bad breath- or body odor-derived smell component.

Also, favorite goods having a smell component and a causative substance thereof may include cigarettes and the like. In the present technology, it is possible to inactivate a corresponding smoking-derived smell component caused by direct smoking or indirect smoking. In the present technology, it is also possible to inactivate a smoking-derived smell component attached to clothes or the like.

The “smell component” as a target of the present technology is not particularly limited, but is preferably a volatile sulfur compound and/or a volatile nitrogen compound.

It is desirable that the corresponding volatile sulfur compound and/or volatile nitrogen compound overlap with a food-derived or smoking-derived smell component. Also, the volatile compound refers to one that is easily volatilized in the atmosphere at a normal temperature and a normal pressure.

Examples of the above volatile sulfur compound may include hydrogen sulfide, mercaptan, disulfide, and the like. The corresponding mercaptan preferably has 1 to 4 carbon atoms, and more preferably has 1 to 3 carbon atoms.

Examples of the corresponding mercaptan may include methyl mercaptan, ethyl mercaptan, propyl mercaptan (aka: 1-propanethiol), allyl mercaptan, and the like.

In the present technology, one type or two or more types may be selected from these.

As for the corresponding volatile nitrogen compound, ammonia, amine, and the like may be exemplified. Preferably, the corresponding amine may include alkylamine-type, pyridine-type (for example, nicotine, etc.), and the like. The corresponding alkylamine-based alkyl group preferably has 1 to 3 carbon atoms, and is more preferably methyl. Examples of the alkylamine-type may include aminomethane (aka: methylamine), dimethylamine, and trimethylamine.

In the present technology, one type or two or more types may be selected from these.

The “smell component” as a target of the present technology is preferably one type or two or more types including hydrogen sulfide, mercaptan, ammonia, and alkylamine-type.

As for mercaptan as a target of the present technology, methyl mercaptan, propyl mercaptan, and allyl mercaptan are preferred in view of being satisfactorily inactivated in the present technology.

The alkylamine-type as a target of the present technology is one type or two or more types including aminomethane, dimethylamine, and trimethylamine.

The “smell component” as a target of the present technology is preferably one type or two or more types including methyl mercaptan, propyl mercaptan, allyl mercaptan, aminomethane, dimethylamine, and trimethylamine.

Among these, methyl mercaptan, propyl mercaptan, allyl mercaptan, trimethylamine, and a mixture of two or more types thereof are desirable in view of being more satisfactorily inactivated in the present technology.

Each preferred concentration in the LPO reaction system of the present technology will be described below in detail.

Regarding the contents of the component (A) lactoperoxidase, the component(B) glucose oxidase, and the component (C) glucose in the composition of the present technology, it is desirable to adjust the concentration of each component (the concentration in the reaction system), in the use, to a concentration described below.

The concentration of the component (A) lactoperoxidase in the LPO reaction system of the present technology is not particularly limited. It is desirable that the lower limit of the concentration of the corresponding component (A) is preferably 0.05 μg/mL or more, more preferably 0.15 μg/mL or more, further preferably 1.5 μg/mL or more, still further preferably 15 μg/mL or more, in view of deodorization rate improvement. Also, it is desirable that the upper limit of the concentration of the corresponding component (A) is preferably 10,000 μg/mL or less, more preferably 250 μg/mL or less, further preferably 150 μg/mL or less in view of the balance between the use amount of the component (A) and the deodorization rate. Also, it is desirable that the range of the concentration of the corresponding component (A) is more preferably 0.1 to 500 μg/mL, further preferably 0.5 to 400 μg/mL, still further preferably 1.2 to 300 μg/mL in view of the cost and the deodorization rate.

The concentration of the component (B) glucose oxidase in the LPO reaction system of the present technology is not particularly limited. It is desirable that the lower limit of the concentration of the corresponding component (B) is preferably 1 μg/mL or more, more preferably 13.5 μg/mL or more, further preferably 15 μg/mL, still further preferably 135 μg/mL or more in view of deodorization rate improvement. Also, it is desirable that the upper limit of the concentration of the corresponding component (B) is preferably 10,000 μg/mL or less, more preferably 3,000 μg/mL or less, further preferably 1,350 μg/mL or less in view of balance between the use amount of the component (A) and the deodorization rate. It is desirable that the range of the concentration of the corresponding component (B) is more preferably 5 to 5,000 μg/mL, further preferably 10 to 4,000 μg/mL, still further preferably 10 to 2,700 μg/mL in view of the cost and the deodorization rate.

The concentration of the component (C) glucose in the LPO reaction system of the present technology is not particularly limited, but the lower limit thereof is preferably 1 μg/mL or more, more preferably 15 μg/mL or more, further preferably 50 μg/m L or more, further preferably 100 μg/mL or more, still further preferably 200 μg/mL or more, and the upper limit thereof is preferably 10,000 μg/mL or less, more preferably 3,000 μg/mL or less, further preferably 1,500 μg/mL or less. It is desirable that the range of the concentration of the corresponding component (C) is more preferably 10 to 10,000 μg/mL, further preferably 10 to 5,000 μg/mL, still further preferably 10 to 3,000 μg/mL in view of the cost, the sweetness reduction, and the deodorization rate.

The concentration of the component (D) thiocyanic acid or the salt thereof in the LPO reaction system of the present technology is preferably 0.1 to 100 mM, more preferably 0.2 to 60 mM, further preferably 0.3 to 20 mM. When the corresponding concentration is not reached, the thiocyanic acid or the salt thereof may be added to the composition of the present technology or may be used in the use region such that the concentration may be reached.

Also, the respective use ratios of the component (A) lactoperoxidase, the component (B) glucose oxidase, and the component (C) glucose in the LPO reaction system of the present technology are not particularly limited, and the above described concentrations may be combined.

Also, when the LPO reaction system of the present technology is used, the mass ratio of the component (A) lactoperoxidase to the component (B) glucose oxidase is not particularly limited, but component (A):component (B) is preferably 1:5 to 15, component (A):component (B) is more preferably 1:7 to 12, or component (A):component (B) is 1:8 to 10, and component (A):component (B) is particularly preferably 1:9.

The component (C) glucose (mass ratio) is not particularly limited, but 0.5 to 1.5 parts by mass may be used with respect to 1 part by mass of the component (B) glucose oxidase. In the present technology, it is particularly preferable that component (A):component(B):component(C)=1:9:10.

The concentration of the “smell component” as a target of the LPO reaction system of the present technology is not particularly limited, but is preferably 500 ppb or less, more preferably 300 ppb or less, further preferably 260 ppb or less.

As illustrated in [Examples] below, the present technology uses the component(A) lactoperoxidase, the component (B) glucose oxidase, and the component (C) glucose, in the presence of thiocyanic acid or the salt thereof.

The components (A) to (C) used in the present technology may be contained as active ingredients of a composition, and these components are effective in inactivating a smell component. Accordingly, bad breath, body odor, or excrement odor may be suppressed or reduced.

It is desirable that the components (A) to (C) of the present technology are used for a generated smell component, and the corresponding components (A) to (C) may be used for inactivating the smell component.

Therefore, the component (A) lactoperoxidase, the component (B) glucose oxidase, and the component (C) glucose of the present technology may be contained as active ingredients in a smell-component inactivating composition, and also, may be contained in a composition that is expected to have a reduction effect or a deodorization effect for an odor caused by a smell component. These various compositions may also be used as preparations.

The component (A) lactoperoxidase, the component (B) glucose oxidase, and the component (C) glucose of the present technology may be used as they are, or may be used after being mixed with a general carrier or diluent which is physiologically or pharmaceutically acceptable.

Further, in the present technology, as necessary, the thiocyanic acid or the salt thereof may be used or present as the component (D) in the composition.

Also, the LPO reaction system of the present technology may be used for various applications such as medicines, foods/drinks and feeds, and various compositions.

Also, the present technology may provide the component (A) lactoperoxidase, the component (B) glucose oxidase, and the component (C) glucose or the use thereof, which is used for the purpose of inactivating the above described smell component.

Also, the component (A) lactoperoxidase, the component (B) glucose oxidase, and the component (C) glucose of the present technology may be used as active ingredients in the method of inactivating the smell component.

Also, the components (A) to (C) of the present technology may be used for producing various preparations or various compositions which have the above described effects or the above described purposes of use.

Also, the component (A) lactoperoxidase, the component (B) glucose oxidase, and the component (C) glucose of the present technology, or the composition containing the corresponding components (A) to (C) may be used for preventing or improving the smell component released to the outside of the body.

Further, in the present technology, as necessary, the thiocyanic acid or the salt thereof may be used or present as the component (D).

Also, in the present technology, each composition containing one type or two or more types of the component (A) lactoperoxidase, the component (B) glucose oxidase, and the component (C) glucose may be produced. Further, in the present technology, as necessary, the thiocyanic acid or the salt thereof may be used or present as the component (D). Then, the use as a composition kit using each composition is possible.

Therefore, the present technology may provide a composition kit including lactoperoxidase, glucose oxidase, and glucose, which inactivates a smell component in the presence of thiocyanic acid or a salt thereof.

It is desirable that the above composition kit contains at least the following (a), (b), (c), or (d). As necessary, the thiocyanic acid or the salt thereof may be used or present as the component (D), or the composition may separately contain the thiocyanic acid or the salt thereof.

(a) a composition containing lactoperoxidase, a composition containing glucose oxidase, and a composition containing glucose;

(b) a composition containing lactoperoxidase and glucose oxidase, and a composition containing glucose;

(c) a composition containing lactoperoxidase, and a composition containing glucose oxidase and glucose; and

(d) a composition containing lactoperoxidase and glucose, and a composition containing glucose oxidase.

In the present technology, when a composition or a preparation is formed using the above components (A) to (C), the content of the above components (A) to (C), as active ingredients, is generally 0.005 to 20 mass %, and preferably 0.005 to 12.5 mass %. Here, an excipient, a binder, a disintegrant, a lubricant, a stabilizer, a flavoring agent, a diluent, and a solvent for injection may be used.

The amount of the composition of the present technology used by a user each time may be properly determined according to the sex, age, condition, etc. of the user.

The composition of the present technology may be used in a amount by which in the above described LPO reaction system of the present technology, the concentration of the component (A) lactoperoxidase, the concentration of the component (B) glucose oxidase, and the concentration of the component(C) glucose may be obtained.

It is desirable to adjust the amount of the composition of the present technology such that in the above described LPO reaction system of the present technology, not only the above concentration ranges of the components (A) to (C), but also the concentration of the thiocyanic acid or the salt thereof may be obtained.

The composition of the present technology may have the form of an aqueous solution composition, or the form of a solid composition.

The composition in the aqueous solution form may be used in such a manner that it is filled into a spray bottle or the like and is sprayed on a target of use. Also, the composition in the solid form may be used in the oral cavity to exert an inactivating effect. In such a case, it is desirable to mold a deodorant of the present technology into a tablet shape or a film shape.

It is desirable to carry out the LPO reaction system of the present technology in the presence of water. Thus, in the use region, the LPO reaction system is preferably placed in a state containing water.

A smell component present in the space of the oral cavity or a smell component passing through the oral cavity is likely to be mixed with saliva due to the movement of the tongue or the like, or the mastication in the oral cavity. By causing the LPO reaction system of the present technology to exist in the oral cavity, the corresponding LPO reaction system is mixed with the saliva and the smell component itself. Accordingly, the corresponding LPO reaction system acts on the smell component itself. Therefore, the smell component itself present in the oral cavity or the smell component itself passing through the oral cavity is inactivated by the LPO reaction system of the present technology. This smell component passing through the oral cavity is derived from a lung and/or derived from a stomach.

The present technology uses the above component (A) lactoperoxidase, the component (B) glucose oxidase, and the component (C) glucose, and an optional component as appropriate, for a subject, and is a method of inactivating a generated smell component itself by the components (A) to (C).

The present technology exerts a high inactivating effect on the smell component in the above described concentration range of each component. Thus, in the present technology, the respective concentrations of lactoperoxidase, glucose oxidase, and glucose when a region where an unpleasant smell component is being produced is treated preferably fall within the above described concentration ranges. Also, it is also desirable that the concentration of thiocyanic acid or a salt thereof falls within the above described concentration range. In general, since the thiocyanic acid is present in the oral cavity, the LPO reaction system of the present technology may satisfactorily progress by the amount of the thiocyanic acid present in the oral cavity and inactivate the smell component, which is also an advantage of the present technology.

A period of a reaction by the LPO reaction system of the present technology is not particularly limited. The corresponding reaction period is preferably 1 min to 1 h, more preferably 5 min to 45 min, further preferably 5 min to 30 min, still further preferably 5 min to 20 min after the start of reaction. Since the advantage of the present technology is not microbial sterilization or enzyme inhibition, inactivation is likely to progress in a relatively short time.

Also, the reaction start of the LPO reaction system of the present technology is not particularly limited. The corresponding reaction is preferably started immediately after a meal to the next day, specifically more preferably 5 min after a meal to 12 h, further preferably 30 min after a meal to 6 h. In the corresponding range, a smell component or a causative substance remains in the body, and is generated as bad breath, body odor or the like.

In the manner of the LPO reaction system of the present technology, components such as the above component (A) to the component (C), and an optional component as appropriate may be sequentially added to a target, or a mixture including the components, that is, a smell component-inactivating composition or composition kit of the present technology may act on a smell component as a target.

Also, since each constituent component of the LPO is empirically highly safe, the use region in the LPO reaction system of the present technology is the oral cavity or the skin from the viewpoint that a generated smell component is likely to exist. The reaction method may include spraying, coating, contact, and the like.

The above components (A) to (C) or the LPO reaction system of the present technology may be used for a pharmaceutical composition or a pharmaceutical application.

As the usage and dose of the present technology, the above described usage and dose may be employed.

Examples of an administration route may include oral administration, transmucosal administration, intranasal administration, rectal administration, and the like. Among these, oral administration (oral ingestion) is preferred.

It is desirable that the administration subject is generally a human, but mammals other than humans, for example, pets such as dogs and cats, and domestic animals such as cows, sheep, and pigs, are also included.

The dosage form (or preparation) may be any form of a solid preparation and a liquid preparation, and examples thereof may include tablets, pills, capsules, discutient, granules, solutions, injections, powder, spray preparations, and the like.

The pharmaceutical composition of the present technology may contain a pharmaceutically acceptable carrier. Such a carrier contains an excipient or a diluent, and examples thereof may include dextran, saccharose, lactose, maltose, xylose, trehalose, mannitol, sorbitol, gelatin, carboxymethyl cellulose, carboxyethyl cellulose, hydroxy propylmethyl cellulose, gum arabic, guar gum, Tragacanth, acrylic acid copolymer, ethanol, saline solution, Ringer's solution, and the like.

In addition to the above carrier, as necessary, additives such as preservatives, stabilizers, binders, pH adjusters, buffers, thickeners, gelling agents, and antioxidants may be added. As these additives, those used in medicine production are preferred.

When a pharmaceutical composition containing the above components (A) to (C) or the LPO reaction system of the present technology, as active ingredients, is produced, production may be performed according to a conventional method in a pharmaceutical technology field, for example, a method described in the Japanese Pharmacopoeia or a method equivalent thereto.

The pharmaceutical composition for inactivating a generated smell component, which is related to the present technology, may be used in combination with other medicines. The medicine to be used in combination may be administered concurrently with administration of the composition of the present technology, before the administration, or at any time after the administration. The dose is not particularly limited, but in a case of commercially available medicines, a dose instructed by a medicine manufacturer is preferred.

[1] Food/Drink Composition and Feed Composition

The present technology may be used for a food/drink composition or a food/drink application, and a feed composition or a feed application. As for the corresponding usage and dose, the above described usage and dose may be employed.

The above components (A) to (C) or the LPO reaction system used in the present technology may be used as active ingredients in foods/drinks for humans or animals, health foods, functional foods, patient foods, enteral nutritional foods, special-purpose foods, health functional foods, foods for specified health uses, functionality labeled foods, nutritionally functional foods, and the like (hereinafter, also referred to as “foods/drinks and the like”), which are to be used for inactivation of the above described smell component, after being blended with these.

For example, the above components (A) to (C) or the LPO reaction system used in the present technology may be used by being added to flour products, instant foods, processed agricultural products, processed seafood products, processed livestock products, milk, dairy products, oils and fats, basic seasonings, complex seasonings or food products, frozen foods, confectioneries, drinks, commercially available foods other than these, tablet confectionaries, liquid foods, feeds (including the use for pets) or the like. As the form of the food/drink, any form such as liquids, pastes, solids, or powder may be used.

The foods/drinks and the like defined in the present technology may also be provided or sold as foods/drinks labeled with a specific application (especially, a health application) or function.

The food/drink composition of the present technology may be provided or sold as a food or a drink labeled with a health application for use in inactivating the above described smell component, or for use in preventing or improving an odor caused by meals or smoking. Examples of such labeling may include labeling such as “those who are worried about bad breath or body odor immediately after eating,” “those who are worried about bad breath or body odor on the next day,” and “after eating plants of the genus Allium such as garlic and green onions.”

The action of “labeling” includes all actions that inform consumers of the above application, and all expressions that correspond to the action of “labeling” of the present invention regardless of the purpose of a label, the contents of a label, and a target object and a medium of labeling as long as the above application may be recalled or analogized.

Also, it is desirable that “labeling” is made by an expression that allows the consumers to directly recognize the above application. Specifically, an action of assignment and delivery of a product or a product packaging related to a food or a drink (on which the above application is described), and display and importation for the assignment or delivery; and an action of displaying or distributing advertisements, a price list, or a transaction document related to the product, on which the above application is described, or providing information having these as contents, on which the above application is described, by an electromagnetic (the Internet, etc.) method may be mentioned.

Meanwhile, as for the label contents, a label permitted by the government or the like, for example, a label that is approved in accordance with various systems specified by the government, and is applied in the form based on such approval, is preferred. Also, it is desirable that such label contents are attached to packaging, containers, catalogs, pamphlets, advertising materials at a sales site such as POP, other documents, and the like.

Also, as for the “label,” labels for health foods, functional foods, enteral nutritional foods, special-purpose foods, health functional foods, foods for specified health uses, nutritionally functional foods, functionality labeled foods, quasi-drugs, etc. may also be exemplified. Among these, particularly, labels approved by the Consumer Affairs Agency, for example, labels approved by systems related to foods for specified health uses, nutritionally functional foods, or functionality labeled foods, or systems similar to these may be exemplified. Specifically, labels for foods for specified health uses, labels for foods for conditional specified health uses, labels to the effect that the structure or function of a body is affected, labels on disease risk reduction, and labels for functionality based on scientific grounds may be exemplified, More specifically, labels for foods for specified health uses (particularly, labels for health applications), which are stipulated in Cabinet Office Ordinance(Cabinet Office Ordinance No. 57, Aug. 31, 2011) concerning permission, etc. for special application labeling prescribed in the Health Promotion Act, and labels similar thereto are typical examples.

Examples of the above food or drink also include fermented foods/drinks, but when the corresponding fermented food/drink is produced, the above components (A) to (C) or the LPO reaction system may be blended. Also, sweeteners such as sucrose, pectin, fruits, fruit juice, agar, gelatin, oils and fats, fragrances, colorants, stabilizers, reducing agents, and the like may be added. Also, the fermented food/drink may be properly filled into a container.

The fermented food/drink obtained by the above described method for producing a fermented food/drink may be properly processed in the same manner as in a normal fermented food/drink.

The fermented food/drink obtained as above contains the above components (A) to (C) or the LPO reaction system, whereby the effect of the present technology may be satisfactorily exerted.

Also, the present technology may be used as an active ingredient of an animal feed to be used for inactivating the above described generated smell component. The present technology may be added to a conventionally known feed in preparation, or may be mixed with a feed raw material so as to produce a new feed.

Examples of the above feed raw material may include: cereals such as corn, wheat, barley, and rye; brans such as wheat bran, barley bran, rice bran, and defatted rice bran; animal feeds such as skim milk powder of production dregs (such as a corn gluten meal or a corn jam meal), whey, fish meal, and bone meal; yeasts such as beer yeast; mineral feeds such as calcium phosphate and calcium carbonate; oils and fats; amino acids; and sugars. Also, examples of the form of the above feed may include pet feeds (pet foods and the like), livestock feeds, fish feeds, and the like.

As described above, the present technology may be used in a wide range of fields such as foods/drinks, food/drink compositions, functional foods, medicines, and feeds.

Also, the present technology may employ the following configurations:

[1] A smell-component inactivating composition comprising lactoperoxidase, glucose oxidase, and glucose, which is used in the presence of thiocyanic acid or a salt thereof. The corresponding composition may suppress or reduce the generation amount of a smell component by inactivating the smell component itself. Accordingly, the corresponding composition may be used for bad breath suppression, body odor suppression, or excrement smell suppression.

[2] A bad breath suppressing composition, a body odor suppressing composition, or an excrement smell suppressing composition, which contains lactoperoxidase, glucose oxidase, and glucose and is used in the presence of thiocyanic acid or a salt thereof. It is desirable that the corresponding composition suppresses or reduces a generated smell component itself.

[3] A smell-component inactivating composition kit comprising lactoperoxidase, glucose oxidase, and glucose, which is used in the presence of thiocyanic acid or a salt thereof.

[4] Lactoperoxidase, glucose oxidase, and glucose for inactivating a smell component in the presence of thiocyanic acid or a salt thereof.

[5] Use of lactoperoxidase, glucose oxidase, and glucose for inactivating a smell component in the presence of thiocyanic acid or a salt thereof.

[6] A method of inactivating a smell component by using lactoperoxidase, glucose oxidase, and glucose, in the presence of thiocyanic acid or a salt thereof. It is possible to suppress or reduce an unpleasant smell (for example, a bad breath, a body odor, an excrement smell or the like) by inactivating the corresponding smell component. The corresponding inactivating method may be for a prevention or therapeutic purpose, or may be for a non-therapeutic purpose.

[7] Use of lactoperoxidase, glucose oxidase, and glucose, in producing a smell-component inactivating composition or a smell-component inactivating composition kit which is used in the presence of thiocyanic acid or a salt thereof.

[8] A prevention method or a treatment method of a symptom generating an unpleasant smell, which includes administering effective amounts of lactoperoxidase, glucose oxidase, and glucose to a human or a patient in need of prevention or treatment. Examples of the corresponding symptom generating the unpleasant smell may include a bad breath, a body odor, and the like, and among these, a bad breath and a body odor are preferred.

[9] A suppression method or a reduction method of any of a bad breath, a body odor, and an excrement smell, which includes administering effective amounts of lactoperoxidase, glucose, oxidase and glucose to a human or a patient in need of prevention or treatment.

[10] In any one of the above [1] to [9], the above smell component is a volatile sulfur compound and/or a volatile nitrogen compound.

[11] In any one of the above [1] to [10], the above smell component is food-derived and/or smoking-derived.

[12] In any one of the above [1] to [11], the above food-derived smell component is a smell component derived from foods containing Allium plants.

[13] In any one of the above [1] to [12], the above smell component is bad breath-derived, body odor-derived, or excretion-derived.

[14] In any one of the above [1] to [13], the above smell component is stomach-derived, lung-derived, or skin-derived.

[15] In any one of the above [1] to [14], the above smell component is one type or two or more types selected from the group including methyl mercaptan, propyl mercaptan, allyl mercaptan, aminomethane, dimethylamine, and trimethylamine.

[16] In any one of the above [1] to [15], the above inactivation is carried out after a meal to the next day.

[17] In any one of the above [1] to [16], the above inactivation is immediate inactivation. A reaction time of the corresponding inactivation is preferably 5 min to 45 min.

[18] In any one of the above [1] to [17], the above smell component is included in foods, clothes, cloth products, sanitary products, excrements or odor-attached products.

[19] In any one of the above [1] to [18], the purpose of use is a non-therapeutic purpose or a bad breath etiquette.

[20] In any one of the above [1] to [19], an application target is an animal, and the corresponding animal is a human or a pet.

### EXAMPLES

The present invention will be described below in more detail with reference to Examples, but the present invention is not limited to these Examples.

### Test Example 1

### Test Example 1-1

This test 1-1 was carried out to examine the action of a composition containing lactoperoxidase, glucose oxidase, and glucose, on sulfur-based smell components (allyl mercaptan and methyl mercaptan).

(1) Preparation of Sample

Raw materials, lactoperoxidase (manufactured by DOMO), glucose oxidase (manufactured by Shin Nihon Chemical Industry Co., Ltd.) and glucose (manufactured by Nakarai Tesque Inc.) were mixed such that the mass ratio of the raw materials may become lactoperoxidase:glucose oxidase:glucose=1:9:10 so as to prepare a LPO composition.

The above LPO composition was dissolved at various concentrations by using 40 mM phosphoric acid buffer (pH 7.7) containing 0.66 mM thiocyanic acid sodium so as to produce LPO solutions. These LPO solutions were employed as test samples. The concentrations of the LPO composition were as follows: 3, 30, 300, and 3,000 μg/LPO solution 1 mL.

Also, epigallocatechin gallate (EGCg, manufactured by Nagara Science Co., Ltd.) known to have a deodorizing effect of allyl mercaptan was dissolved at various concentrations in 40 mM phosphoric acid buffer (pH 7.7) containing 0.66 mM thiocyanic acid sodium so as to prepare EGCg solutions. These were employed as comparative samples. The concentrations of EGCg were set as follows: 3, 30, 300, and 3,000 μg/mL as in the test samples.

(2) Inactivation Confirmation Test of Smell Component

A vial bottle in which the LPO solution produced in the above (1) or 10 mL was collected was quickly sealed after allyl mercaptan (manufactured by Tokyo Chemical Industry Co., Ltd.) was added thereto to a concentration of 250 ppb. After warming at 37° C. for 30 min, 2 mL of headspace gas was collected and injected to a detector for gas chromatography (GC7890B, manufactured by Agilent), and the content of allyl mercaptan was analyzed. Regarding the amount of allyl mercaptan, allyl mercaptan was quantified from a calibration curve created in advance.

Also, the same test was carried out by changing allyl mercaptan to methyl mercaptan (manufactured by Wako Pure Chemical Industries, Ltd.).

<Gas Chromatography Conditions>
column: Agilent DB-1 (60 m×0.32 mm×5 um)
detector: PFPD
carrier gas: He
flow velocity: 1.7 mL/min
inlet temperature: 230° C.
oven temperature: 35° C.<0.1° C./min<36° C.<15° C./rain<240° C. (10 min)
detector temperature: 260° C.

(3) Result

As a result, the deodorization rates of allyl mercaptan and methyl mercaptan are noted in Tables 1 and 2 below. The deodorization rate indicates the ratio of the component amount in the test sample or the comparative sample, with respect to each component amount in headspace detection of a solution (the concentration of allyl mercaptan or methyl mercaptan was 250 ppb) not containing the composition of the LPO reaction system or EGCg.

TABLE 1
Table 1. Deodorizing effect on allyl mercaptan

| | |
|---|---|
| Sample Concentration | Deodorization Rate (%) |

| (μg/mL) | LPO Composition | Control (EGCg) |
|---|---|---|
| 3,000 | 100.0 | 24.3 |
| 300 | 100.0 | 21.7 |
| 30 | 99.6 | 13.2 |
| 3 | 65.3 | 16.4 |

TABLE 2
Table 2. Deodorizing effect on methyl mercaptan

| | |
|---|---|
| Sample Concentration | Deodorization Rate (%) |

| (μg/mL) | LPO Composition | Control (EGCg) |
|---|---|---|
| 3,000 | 100.0 | 43.0 |
| 300 | 100.0 | 14.6 |
| 30 | 100.0 | 14.5 |
| 3 | 83.3 | 4.1 |

### Test Example 1-2

This test 1-2 was carried out to examine the action on propyl mercaptan, regarding whether there is a deodorizing effect for sulfur-based smell components other than the sulfur-based smell components such as allyl mercaptan and methyl mercaptan. The same test as the above test example 1-1 was carried out except that the concentrations of the LPO composition were set as follows: 30, 300, and 3,000 μg/LPO solution 1 mL, and allyl mercaptan was replaced with propyl mercaptan (manufactured by Tokyo Chemical Industry Co., Ltd.).

As a result, the deodorization rates of propyl mercaptan are noted in Table 3 below. The deodorization rate indicates the ratio of the component amount in the test sample or the comparative sample, with respect to each component amount in headspace detection of a solution (propyl mercaptan concentration was 250 ppb) not containing the composition of the LPO reaction system or EGCg.

TABLE 3
Table 3. Deodorizing effect on propyl mercaptan

| | |
|---|---|
| Sample Concentration | Deodorization Rate (%) |

| (μg/mL) | LPO Composition | Control (EGCg) |
|---|---|---|
| 3,000 | 100.0 | 16.6 |
| 300 | 100.0 | 15.8 |
| 30 | 100.0 | 13.8 |

As a result of the above test example 1-1 and the test example 1-2, in the comparative sample, although the deodorizing effect increased depending on the concentration of epigallocatechin gallate, even at the highest concentration (3,000 μg/mL) of epigallocatechin gallate, the deodorization rate remained at 24.3%.

In contrast, the test sample also similarly exhibited the concentration-dependent deodorizing effect, but exhibited a higher effect at a lower concentration than the comparative sample. It was confirmed that the test sample has a high deodorizing effect that exhibits a deodorization rate of 36% at a concentration of only 3 μg/mL, and a deodorization rate of almost 100% at a concentration of 30 μg/mL. Also, regarding vial bottles in which the deodorization rates became 100% due to the use of the LPO compositions in Tables 1 to 3, when the experimenter sniffed and checked whether there was a smell by opening the vial bottles, no smell was recognized.

### Test Example 2

This test was carried out to examine the action of a composition containing lactoperoxidase, glucose oxidase, and glucose, on a nitrogen-based smell component (trimethylamine).

The test was carried out in the same procedure as in the test example 1 by replacing a smell component with trimethylamine. Specifically, 20 mL of the LPO solution prepared in (1) of the test example 1 was collected in a 1 L-capacity Erlenmeyer flask, and then sealed and warmed at 37° C. for 30 min after 0.5 mL of 0.3% trimethylamine solution was further added. Then, 100 mL of headspace gas was collected by a detector (Gastec corporation, detector tube (trimethylamine; No. 3M)), and the concentration of trimethylamine was measured.

The results are noted in Table 4 below. It was confirmed that the test sample exhibits a deodorizing effect on trimethylamine in a concentration-dependent manner. That is, it was found that the LPO composition also exhibits a deodorizing effect on a nitrogen-based smell component.

TABLE 4
Table 4. Deodorizing effect on trimethylamine

| Sample Concentration | Deodorization Rate(%) |
|---|---|
| (μg/mL) | LPO Composition |
| 3,000 | 99.1 |
| 1,000 | 61.3 |
| 300 | 43.9 |

From the above, it was found that the LPO composition has an effect of inactivating various smell components. These various smell components or their causative substances are included especially in plants of the genus Allium and cigarettes.

Therefore, the composition of the LPO reaction system of the present technology targets and inactivates a generated smell component caused by oral ingestion of foods or smoking, and thus is useful for applications such as suppression or reduction of bad breath, body odor, or the like. The present technology has a novelty in that a smell component generating step is not targeted, but a smell component itself that is generated and remains in the body is targeted.

1. A composition for inactivating a smell component comprising lactoperoxidase, glucose oxidase, and glucose, and optionally thiocyanic acid or a salt thereof.

2. The composition according to claim 1, wherein the smell component is a volatile sulfur compound and/or a volatile nitrogen compound.

3. The composition according to claim 1, wherein the smell component is food-derived and/or smoking-derived.

4. The composition according to claim 3, wherein the food-derived smell component is derived from a food comprising a plant of the genus Allium.

5. A smell component-inactivating composition kit comprising lactoperoxidase, glucose oxidase, and glucose, and optionally thiocyanic acid or a salt thereof.

6. The smell component-inactivating composition kit according to claim 5, wherein the composition kit comprises a composition selected from the group consisting of:
(a) a composition comprising lactoperoxidase, a composition containing glucose oxidase, and a composition containing glucose;
(b) a composition comprising lactoperoxidase and glucose oxidase, and a composition comprising glucose;
(c) a composition comprising lactoperoxidase, and a composition comprising glucose oxidase and glucose;
(d) a composition comprising lactoperoxidase and glucose, and a composition comprising glucose oxidase; and
(e) combinations thereof.

7. A method of inactivating a smell component by using lactoperoxidase, glucose oxidase, and glucose, in the presence of thiocyanic acid or a salt thereof.
